(19) United States
(12) Patent Application Publication
(10) Pub. No.: US 2015/0133555 A1
(43) Pub. Date: May 14, 2015

(54) COMPOSITIONS FOR AMELIORATING A REDUCED HIGHER BRAIN FUNCTION RESULTING FROM ORGANIC BRAIN LESIONS

(71) Applicants: SUNTORY HOLDINGS LIMITED, Osaka (JP); NATIONAL UNIVERSITY CORPORATION KANAZAWA UNIVERSITY, Ishikawa (JP)

(72) Inventors: Yoshiyuki ISHIKURA, Osaka (JP); Tetsumori YAMASHIMA, Ishikawa (JP)

(73) Assignees: SUNTORY HOLDINGS LIMITED, Osaka (JP); NATIONAL UNIVERSITY CORPORATION KANAZAWA UNIVERSITY, Ishikawa (JP)

(21) Appl. No.: 14/601,317

(22) Filed: Jan. 21, 2015

Related U.S. Application Data
(63) Continuation of application No. 11/922,559, filed on Dec. 20, 2007, now abandoned.

(30) Foreign Application Priority Data
Jun. 30, 2005 (JP) 2005-191624

(51) Int. Cl. A61K 31/202

(52) U.S. Cl. CPC A61K 31/202
USPC 514/560

(57) ABSTRACT

A composition that has an activity of ameliorating reduced higher brain functions resulting from organic brain lesions, said composition comprising arachidonic acid and/or a compound having arachidonic acid as a constituent fatty acid as well as docosahexaenoic acid and/or a compound having docosahexaenoic acid as a constituent fatty acid.

### TECHNICAL FIELD

The present invention relates to a composition having an activity of ameliorating a reduced higher brain function.

### BACKGROUND ART

Higher brain functions refer to such brain functions as attention, memory, perception, language, and calculation, and higher brain dysfunction are noted when organic brain lesions have occurred due to ischemic stroke such as cerebral infarction and transient cerebral ischemic attack, hemorrhagic stroke such as cerebral hemorrhage and subarachnoid hemorrhage, and lesional diseases such as cerebral concussion and cerebral contusion. Higher brain dysfunction includes, for example, hemiasomatognosia, topographical disorder, agnosia, aphasia, dysmnesia, apraxia, disturbance of attention, performance dysfunction, and dysfunction of action and emotion.

With regard to these higher brain dysfunction, various therapeutic agents are being studied and developed. Most of them, however, are symtomatic to causes of organic brain lesions and unfortunately there have been found no compounds that ameliorate or enhance the reduced higher brain function. As methods of judging and evaluating the ameliorating activity of higher brain dysfunction, there can be mentioned the Mini Mental State Examination (MMSE), the Revised version of the Hasegawa's Dementia Scale (HDS-R) and the Wechsler Adult Intelligence Scale-Revised (WAIS-R). There were drawbacks, however, in that MMSE and HDS-R lack both precision and reproducibility in evaluating the amelioration or enhancement of higher brain function and WAIS-R took too much time and labor. Accordingly, in order to research and develop pharmaceutical agents for the purpose of objectively ameliorating or enhancing higher brain function, an effective means for objectively evaluating a higher brain function was needed.

Recently, the RBANS neuropsychological test is attracting attention as a method of evaluating higher brain function with high precision and reproducibility. The RBANS neuropsychological test is an abbreviation of Repeatable Battery for the Assessment of Neuropsychological Status, was developed by Randolph of the USA, and is psychological test problems that can be repeated and evaluated for a short time of 30 minutes. This RBANS neuropsychological test is very effective for early diagnosis, follow-up and judgment of therapeutic effect of psychoneurotic diseases as represented by senile dementia and schizophrenia, and is believed to be also useful for judging cerebrovascular disorders and late effects of head injuries, i.e. higher brain, dysfunction [Tetsumori Yamajima et al., Cranial Nerves (Nousinkei) Vol. 54, 463-471 (2002)]. Thus the RBANS neuropsychological test made it possible to judge the higher brain functions of humans with high reproducibility in a short period of time.

The brain is a tissue which is similar to a lipid mass, and one third, for example, of white matter and one fourth of cinerea is occupied by phospholipid. Higher unsaturated fatty acids in the phospholipid constituting various cell membranes of the brain are dominantly arachidonic acid and docosahexaenoic acid. However, these arachidonic acid and docosahexaenoic acid cannot be synthesized de novo in animal bodies, and must be ingested directly or indirectly in the diet (linolenic acid is a precursor of arachidonic acid, and α-linolenic acid is a precursor of docosahexaenoic acid). Thus, attention has been centered on the enhanced ability of learning and memory and prevention of and recovery from senile dementia due to arachidonic acid and docosahexaenoic acid.

For docosahexaenoic acid, there is an ample source as fish oil, and thus a variety of studies have been made on the amelioration of brain functions with a result that patents have been filed on a learning ability enhancing agent, a memory enhancing agent, a dementia preventing agent, a dementia treating agent, anti-dementia agent, or functional foods having an ability of ameliorating reduced brain function (Japanese Unexamined Patent Publication (Kokai) No. 7-82146, Japanese Unexamined Patent Publication (Kokai) No. 5-117147, Japanese Unexamined Patent Publication (Kokai) No. 2-49723). Regarding arachidonic acid and/or a compound having arachidonic acid as a constituent fatty acid, it was demonstrated recently in Japanese Unexamined Patent Publication (Kokai) No. 2003-048831 “A composition having an ability of preventing or ameliorating symptoms or diseases resulting from reduced brain function” that a decrease in learning ability associated with aging was ameliorated by the administration of arachidonic acid and/or a compound having arachidonic acid as a constituent fatty acid in a test in which an aged animal was subjected to a Morris water maze test.

Miyanaga et al. of Gunma University Faculty of Medicine have found that when capsules containing 2400 mg of docosahexaenoic acid (DHA) were orally administered to 26 normal healthy individuals and event-related potential (P300) which correlates with perception response was measured before and 2 hours after the administration, the latent time was significantly curtailed and amplitude was significantly enhanced [Kazuo Miyanaga, Science of Eating (Shokuno Kagaku), pp. 84-96 (1999)]. However, this was not a comparison with placebo and no correlation was observed between docosahexaenoic acid (DHA) concentrations in the blood and the result of P300; furthermore, in a prolonged administration test in which capsules containing 900 mg of docosahexaenoic acid (DHA) were orally administered to 97 normal elderly individuals every day for six months, no changes were noted in P300, which is a result simply insufficient to demonstrate the effectiveness of docosahexaenoic acid (DHA).

Miyanaga et al. also reported that in 10 of 13 cases of cerebrovascular dementia and all five cases of Alzheimer's dementia who received the oral administration of capsules containing 700-1400 mg of docosahexaenoic acid (DHA) per day for six months, the effect of a slight amelioration or better was noted [Kazuo Miyanaga, Clinical Nutrition (Rinsho Eiyo), 881-901 (1995)]. However, this was a mere enhancement in communication of will and spontaneity, amelioration of delirium, poriomania, depressive conditions, and gait disturbance, and not of enhancement in the memory and learning ability.

Thus, despite the expectations of an ameliorating activity of memory and learning abilities for arachidonic acid and docosahexaenoic acid, there nave only been demonstrated the amelioration of event-related potential in normal healthy elderly individuals and the amelioration of emotional and behavioral disorders in patients with Alzheimer's disease, and it is not clear, at all, whether they exhibit an ameliorating or enhancing activity of reduced higher brain functions resulting from organic brain lesions.

Thus, there is a strong need for the development of compounds that can ameliorate reduced higher brain functions resulting from organic brain lesions, and of compounds with less side effects having excellent application into pharmaceutical drugs and even foods.

Patent document 1: Japanese Unexamined Patent Publication (Kokai) No. 2003-048831

Non-patent document 1: Kazuo Miyanaga, Science of Eating (Shokuno Kagaku), pp. 84-96 (1999)

Non-patent document 2: Kazuo Miyanaga, Clinical Nutrition (Rinsho Eiyo), 881-901 (1995)

### DISCLOSURE OF THE INVENTION

Thus, it is an object of the present invention to provide a food and a drink that have an activity of ameliorating reduced higher brain functions resulting from organic brain lesions, and a method of producing them, said food and drink comprising, as an active ingredient, arachidonic acid and/or a compound having arachidonic acid as a constituent fatty acid as well as docosahexaenoic acid and/or a compound having docosahexaenoic acid as a constituent fatty acid. More specifically, it is an object of the present invention to provide a food and a drink that have an activity of ameliorating reduced higher brain functions resulting from organic brain lesions, and a method of producing them, said food and drink comprising, as an active ingredient, at least one selected from the group consisting of: arachidonic acid and docosahexaenoic acid; an alcohol ester of arachidonic acid or docosahexaenoic acid; and a triglyceride, a phospholipid and a glycolipid wherein part or all of the constituent fatty acids is arachidonic acid and/or docosahexaenoic acid, and a method of producing them.

After intensive and extensive research to elucidate the effect, of ameliorating reduced higher brain functions resulting from organic brain lesions, of arachidonic acid and/or a compound having arachidonic acid as a constituent fatty acid as well as docosahexaenoic acid and/or a compound having docosahexaenoic acid as a constituent fatty acid, the present inventors have surprisingly demonstrated, in humans, the effect of arachidonic acid and/or a compound having arachidonic acid as an active ingredient as well as docosahexaenoic acid, and/or a compound having docosahexaenoic acid as a constituent fatty acid on patients with higher brain dysfunction due to organic brain lesions by evaluation with the RBANS neuropsychological test as an index.

Thus, the present invention provides a food and a drink that have an activity of ameliorating reduced higher brain functions resulting from organic brain lesions, said food and drink comprising, as an active ingredient, arachidonic acid and/or a compound having arachidonic acid as a constituent fatty acid as well as docosahexaenoic acid and/or a compound having docosahexaenoic acid as a constituent fatty acid. More specifically, the present invention provides a food and a drink that have an activity of ameliorating reduced higher brain functions resulting from organic brain lesions, said food and drink comprising, as an active ingredient, at least one selected from the group consisting of: arachidonic acid and docosahexaenoic acid; an alcohol ester of arachidonic acid or docosahexaenoic acid; and a triglyceride, a phospholipid and a glycolipid wherein part or ail of the constituent fatty acids is arachidonic acid and/or docosahexaenoic acid, and a method of producing them.

The present invention can provide a food and a drink that have an activity of ameliorating reduced higher brain functions resulting from organic brain lesions, said food and drink comprising, as an active ingredient, arachidonic acid and/or a compound having arachidonic acid as a constituent fatty acid as well as docosahexaenoic acid and/or a compound having docosahexaenoic acid as a constituent fatty acid, and a method of producing them and, therefore, is very useful for humans in modern society.

### BRIEF EXPLANATION OF THE DRAWINGS

FIG. 1 is a drawing that shows the effect of an arachidonic acid- and docosahexaenoic acid-containing oil on higher brain dysfunction (immediate memory and delayed memory) of patients with organic brain lesions as measured by the RBANS neuropsychological test.

### BEST MODE FOR CARRYING OUT TEE INVENTION

The present invention relates to a food and a drink that have an activity of ameliorating reduced higher brain functions resulting from organic brain lesions, said food and drink comprising, as an active ingredient, arachidonic acid and/or a compound having arachidonic acid as a constituent fatty acid as well as docosahexaenoic acid and/or a compound having docosahexaenoic acid as a constituent fatty acid, and a method of producing them.

Reduced higher brain functions resulting from organic brain lesions includes hemiasomatognosia, topographical disorder, agnosia, aphasia, dysmnesia, apraxia, disturbance of attention, performance dysfunction, and disorders of action and emotion and the like resulting from organic brain lesions caused by ischemic stroke such as cerebral infarction and transient cerebral ischemic attack, hemorrhagic stroke such as cerebral hemorrhage and subarachnoid hemorrhage, and traumatic diseases such as cerebral concussion and cerebral contusion. However, these disorders are not limiting, and any condition associated with reduced higher brain functions resulting from organic brain lesions is included.

The active ingredient of the present invention is arachidonic acid and/or docosahexaenoic acid, and all compounds having arachidonic acid and/or docosahexaenoic acid, as a constituent fatty acid can be used. Compounds having arachidonic acid and/or docosahexaenoic acid as a constituent fatty acid include, for example, arachidonic acid salts and/or docosahexaenoic acid salts such as a calcium salt and a sodium salt. Alcohol esters of arachidonic acid and/or docosahexaenoic acid include, for example, an arachidonic acid methyl ester and a docosahexaenoic acid ethyl ester. Also, there can be used triglycerides, phospholipids, glycolipids etc. in which part or all of the constituent fatty acids is arachidonic acid and/or docosahexaenoic acid.

When an application into foods is contemplated, arachidonic acid and/or docosahexaenoic acid is preferably in the form of a triglyceride or a phospholipid, specifically a triglyceride.

Thus, in accordance with the present invention, there can be used a triglyceride containing a triglyceride (a triglyceride containing arachidonic acid and/or docosahexaenoic acid) in which part or all of the constituent fatty acid which is the active ingredient of the present invention is arachidonic acid and/or docosahexaenoic acid. As an arachidonic acid- and/or docosahexaenoic acid-containing triglyceride, an oil (triglyceride) in which the ratio of arachidonic acid or docosahexaenoic acid in the total fatty acids constituting the triglyceride is 5 (w/w) % or greater, preferably 10 (w/w) % or greater, more preferably 20 (w/w) % or greater, and still more preferably 30 (w/w) % or greater, are the desired form when applied to foods. Thus, in accordance with the present invention, all of arachidonic acid- and/or docosahexaenoic acid-containing oils (triglycerides) that are produced by cultivating microorganisms having the ability of producing them may be used.

Microorganisms that have an ability of producing arachidonic acid-containing oils (triglycerides) include, for example, microorganisms belonging to genus Mortierella, genus Conidiobolus, genus Pythium, genus Phytophthora, genus Penicillium, genus Cladosporium, genus Mucor, genus Fusarium, genus Aspergillus, genus Rhodotorula, genus Entomophthora, genus Echinosporangium and genus Saprolegnia.

As microorganisms belonging to genus Mortierella subgenus Mortierella, there can be mentioned Mortierella elongata, Mortierella exigua, Mortierella hygrophila, Mortierella alpina, and the like. Specifically there can be mentioned strains Mortierella elongata IFO8570, Mortierella exigua IFO8571, Mortierella hygrophila IFO5941, Mortierella alpina IFO8S68, ATCC16266, ATCC32221, ATCC42430, CBS219.35, CBS224.37, CBS250.53, CBS343.66, CBS527.72, CRS529.72, CBS608.70, and CBS754.68, and the like.

These strains are all available without limitations from the Institute of Fermentation (IFO) in Osaka City, Japan, the American Type Culture Collection (ATCC) in the U.S.A., and Centrralbureau voor Schimmelcultures (CBS). It is also possible to use Mortierella elongate SAM0219 (FERM P-8703) (FERM BP-1239), a microbial strain isolated from the soil by the study group of the present invention.

In methods of producing foods and drinks that have an activity of improving reduced higher brain functions, a compound having arachidonic acid and/or docosahexaenoic acid as a constituent fatty acid can be used alone or blended with a raw material for foods and drinks that contains virtually no arachidonic acid and/or docosahexaenoic acid, or that contains a very small amount, if any, of arachidonic acid and/or docosahexaenoic acid. The very small amount as used herein means the amount that even when the raw material for foods and drinks contains arachidonic acid and/or docosahexaenoic acid and a food composition having the material blended therein is ingested by humans, it does not reach the daily intake (described hereinafter) of arachidonic acid per day of the present invention.

In the case of a triglyceride in which part or all of the constituent fatty acids is arachidonic acid and/or docosahexaenoic acid, oils (triglycerides) have numerous potentials in application, and can be used as raw materials and additives for foods, beverages, cosmetics, pharmaceuticals, and quasi drugs. The intended use and the amount used have no limitation.

For example, as food compositions there can be mentioned functional foods, nutrient supplements, foods for specified health uses, modified milk for premature infants, modified milk for babies, baby foods, foods for pregnant, women or foods for the elderly people and the like, in addition to general foods. As examples of foods containing oils, there can be mentioned natural foods that originally contain oils such as meat, fish and nuts, foods to which oils are added at the time of cooking such as soup, foods for which oils are used as a heat medium such as donuts, fatty foods such as butter, processed foods to which oils are added at the time of processing such as cookies, or foods to which oils are sprayed or applied at the finish of processing such as hard biscuits, and the like. Furthermore, oils may be added to agricultural foods, fermented foods, livestock food products, aquatic foods, or beverages that contain no oils. Furthermore, they may be in the form of functional foods pharmaceuticals, and quasi drugs, and may also be a processed form, such as enteral foods, powders, granules, troches, oral liquids, suspensions, emulsions, syrups and the like. The product of the present invention may have attached a label indicating that it has an activity of improving reduced higher brain function resulting from organic brain lesions and that said product comprises a compound having arachidonic acid as a constituent fatty acid.

The composition of the present invention may contain various carriers and additives that are generally used for foods or drinks, pharmaceuticals or quasi drugs in addition to the active ingredient of the present invention. Specifically it is preferred to contain antioxidants in order to prevent oxidation of the active ingredient of the present invention. As antioxidants, there can be mentioned naturally occurring antioxidants such as tocopherols, flavone derivatives, phyllodulcins, kojic acid, gallic acid derivatives, catechins, fuki acid, gossypol, pyrazine derivatives, sasamol, guaiacol, guaiac acid, p-coumaric acid, nordihydroguaiatic acid, sterols, terpenes, nucleobases, carotenoids and lignins, and synthetic antioxidants represented by ascorbate-palmitate ester, ascorbate-stearate ester, butyl hydroxy anisole (BHA), butyl hydroxy toluene (BHT), mono-t-butyl hydroxy quinone (TBHQ), and 4-hydroxymethyl-2,6-di-t-butyl phenol (HMBP).

In tocopherols, α-tocopherol, β-tocopherol, γ-tocopherol, δ-tocopherol, ε-tocopherol, ζ-tocopherol, η-tocopherol, and tocopherol esters (tocopherol acetates etc.) may be mentioned as related compounds. Furthermore, in carotenoids, there can be mentioned, for example, β-carotene, canthaxantin, astaxanthin and the like.

As carriers, the composition of the present invention can include, in addition to the active ingredient of the present invention, various carriers, extender agents, diluents, bulking agents, dispersants, excipients, binding solvents (for example water, ethanol, vegetable oils), dissolution adjuvants, buffers, dissolution-promoting agents, gelling agents, suspending agents, wheat flour, rice flour, starch, corn starch, polysaccharides, milk proteins, collagen, rice oils, lecithin and the like. As additives, it can include, but is not limited to, vitamins, sweeteners, organic acids, coloring agents, perfumes, anti-wetting agents, fibers, electrolytes, minerals, nutrients, antioxidants, preservatives, flavoring agents, wetting agents, extracts of natural foods, vegetable extracts and the like.

The main pharmaceutically active ingredient of arachidonic acid and a compound which has an arachidonic acid and/or docosahexaenoic acid as a constituent fatty acid is arachidonic acid and/or docosahexaenoic acid. It is reported that the daily dietary intake of arachidonic acid is 0.14 g in the Kanto area and 0.19-0.20 g in the Kansai area, and the daily dietary intake of docosahexaenoic acid is 0.37-0.38 g in the Kanto area and 0.69-0.82g in the Kansai area [Sisitsu Eiyougaku (Lipid Nutrition) 4: 73-82, (1995)], and a significant amount of, or more, arachidonic acid and/or docosahexaenoic acid must be ingested. Thus, the daily intake of arachidonic acid and/or docosahexaenoic acid and a compound having arachidonic acid, and/or docosahexaenoic acid as a constituent fatty acid by a human adult (having, for example, a body weight of 60 kg) is, in terms of arachidonic acid and/or docosahexaenoic acid, 0.001 g-20 g, preferably 0.01 g-10 g, more preferably 0.05 g-5 g, and most preferably 0.1 g-2 g.

When the active ingredient of the present invention is actually applied into foods or drinks, the absolute amount of arachidonic acid and/or docosahexaenoic acid that is blended with the foods or drinks is important. However, when, a triglyceride containing a triglyceride in which part or ail of the constituent fatty acids is arachidonic acid and/or docosahexaenoic acid is blended into a food, it is blended to 0.001% by weight or more, preferably 0.01% by weight or more, and more preferably 0.1% by weight or more as arachidonic acid, because the absolute amount to be blended to a food or drink may vary with the amount ingested of the blended food or a drink.

When the composition of the present invention is used as a pharmaceutical product, it can be produced according to a method commonly used in the field of pharmacy, for example a method described in the Japanese Pharmacopoeia or a method in conformity therewith.

When the composition of the present invention is used as a pharmaceutical product, the amount blended of the active ingredient in the composition is not specifically limited and can be used at a suitable blend ratio as appropriate as long as the purpose of the present invention is attained.

When the composition of the present invention is used as a pharmaceutical product, preferably it is administered in a unit dosage form, and specifically it is orally administered.

Dosage of the composition of the present invention may differ with age, body weight, disease condition, administration frequency etc., and the daily dosage of a compound having arachidonic acid and/or docosahexaenoic acid as a constituent fatty acid, of the present invention for an adult (about 60 kg), in terms of arachidonic acid and/or docosahexaenoic acid, is generally about 0.001 g-20 g, preferably 0.01 g-10 g, more preferably 0.05 g-5 g, and most preferably 0.1 g-2 g which may be daily administered in 1-3 divided doses.

The major fatty acid of the phospholipids in the cell membrane of the brain are arachidonic acid and docosahexaenoic acid, and, considering the balance, the composition of the present invention is preferably a combination of arachidonic acid and docosahexaenoic acid. Generally, arachidonic acid (n-6 series unsaturated fatty acid) and docosahexaenoic acid (n-3 series unsaturated fatty acid) are each biosynthesized from linoleic acid and α-linolenic acid by the same enzyme. Thus, when arachidonic acid is administered alone, it inhibits the biosynthesis of docosahexaenoic acid. Conversely, when docosahexaenoic acid is administered alone, it inhibits the biosynthesis of arachidonic acid. In order to prevent these drawbacks, it is preferred to take arachidonic acid and docosahexaenoic acid in combination. Also, as the ratio of eicosapentaenoic acid in the phospholipid membrane of the brain is very low, the combination of arachidonic acid and docosahexaenoic acid with little eicosapentaenoic acid is preferred. In the combination of arachidonic acid and docosahexaenoic acid, the ratio (weight) of arachidonic acid/docosahexaenoic acid is in the range of 0.1-15, and preferably in the range of 0.25-10. Furthermore, foods and drinks in which eicosapentaenoic acid has been blended at an amount not exceeding one fifth (weight ratio) of arachidonic acid are preferred.

### EXAMPLES

The present invention will now be explained in more details with reference to specific examples. It should be noted, however, that the present invention is not limited by these examples in any way.

### Example 1

### A method of Producing a Triglyceride Containing Arachidonic Acid as a Constituent Fatty Acid

As the arachidonic acid-producing microorganism, Mortierella alpina was used. Six kiloliters of a medium containing 1.8% glucose, 3.1% defatted soy bean flour, 1.2% soy bean oil, 0.3% KH₂PO₄, 0.1% Na₂SO₄, 0.05% CaCl₂.2H₂O, and 0.05% MgCl₂.6H₂O was prepared in a 10 kL culture tank, and the starting pH was adjusted to 6.0. 30 L of a preculture was inoculated, and was subjected to an aerated stirring culture at a condition of 26° C., an aeration rate of 360 m³/h, a tank pressure of 200 kPa for 8 days. The agitation rate was adjusted so as to maintain the concentration of dissolved oxygen at 10-15 ppm. Furthermore, the glucose concentration was maintained to be within 1-2.5% by the draining method until day 4, and within 0.5-1% thereafter (the above % means weight (W/V) %).

After the completion of culturing, filtration and drying was conducted to obtain a mycelia containing triglycerides having arachidonic acid as a constituent fatty acid, and by hexane extraction of the mycelia obtained, oil was extracted, and, via a purification process (degumming, deacidification, deodorization, depigmentation), 220 Kg of an arachidonic acid-containing triglyceride (triglyceride in which part or all of the constituent fatty acid is arachidonic acid) was obtained. The oil (triglyceride) obtained was methylesterified, and the fatty acid methyl ester obtained was analyzed by gas chromatography, which indicated that the ratio of arachidonic acid in the total fatty acids was 27.84% by weight. Furthermore, the above arachidonic acid-containing oil (triglyceride) was ethylesterified, and from the fatty acid ethyl ester mixture containing 27% by weight of the arachidonic acid ethyl ester, 99% by weight of arachidonic acid ethyl ester separated and purified by a standard high performance liquid chromatography.

### Example 2

### Production of Test Capsules

Water was added to 100 parts by weight of gelatin and 35 parts by weight of food additive glycerin and dissolved at 50-60° C. to prepare a gelatin coat with a viscosity of 2000 cp. Then 60% by weight of arachidonic acid-containing oil (triglyceride) obtained in Example 1 and 40% by weight of fish oil (tuna oil: the ratio of docosahexaenoic acid in the total fatty acids is 40.5%) were mixed, to which vitamin E oil was mixed to 0.05% by weight to prepare content 1. The content 1 was used to form capsules, which were then dried to prepare soft capsules containing 240 mg of the content per capsule. As placebo capsules for tests on humans, soft capsules in which the content was replaced with olive oil (vitamin E oil was mixed at 0.05% by weight to olive oil) were prepared at the same time.

### Example 3

### Study on the Effect of Ingestion of Arachidonic Acid- and Docosahexaenoic Acid-Containing Edible Oil Capsules on Higher Brain, Function of Patients with Organic Brain Lesions

As the RBANS neuropsychological test (Repeatable Battery for the Assessment of Neuropsychological Status), a Japanese version of RBANS neuropsychological test [Cranial Nerves (Nousinkei) Vol. 54, 463-471 (2002)] which is a Japanese translation of the method of Randolph, developer of the method [J Clin Exp Neuropsychol Vol. 20 310-319 (1938)] was used. Thus, five perception regions of immediate memory, visual space/construction, language, attention, and delayed memory] were evaluated by 12 sub-tests. The study of the present invention on humans was conducted under careful consideration and pursuant to the Helsinki Declaration.

After a briefing on the consent of entry into the study, six patients (3 patients with brain contusion, 3 patients with cerebral infarction: the degree of higher brain dysfunction was stabilized in all of them) with organic brain lesions, who consented, were subjected to the RBANS neuropsychological test. From the following day, in order to ingest 240 mg each of arachidonic acid and docosahexaenoic acid per day, 6 capsules containing an arachidonic acid- and docosahexaenoic acid-containing edible oil (40 mg/capsule each in terms of arachidonic acid and docosahexaenoic acid) prepared as in Example 2 were orally administered for 3 months. After capsule intake, the RBANS neuropsychological test was also performed, and raw scores were compared with the result before administration on five perception regions of immediate memory, visual space/construction, language, attention, and delayed memory. In the RBANS neuropsychological test performed on normal healthy elderly individuals, no differences due to repeat experiments were noted, and thus it is estimated that there is no learning effect.

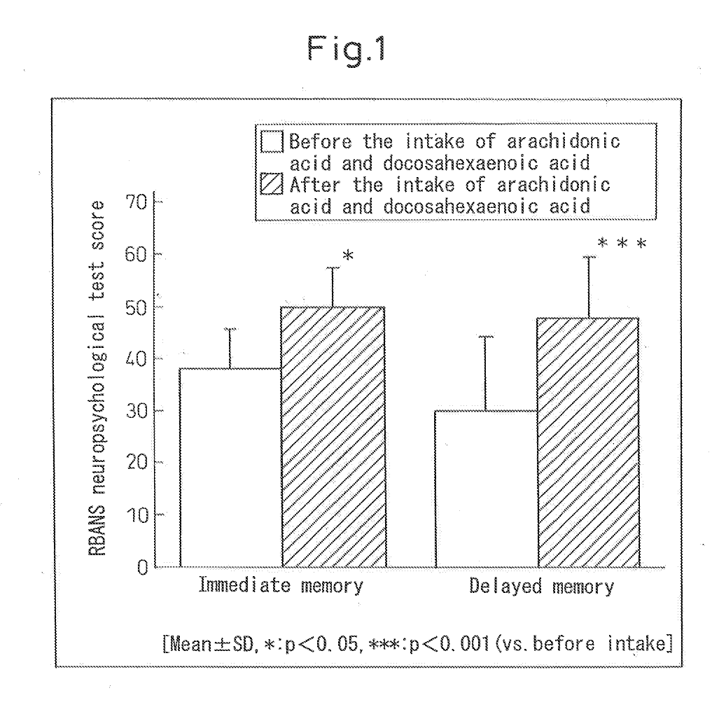

Changes in raw scores of immediate memory and delayed memory before and after capsule intake are shown in FIG. 1. It was demonstrated that by ingesting arachidonic acid- and docosahexaenoic acid-containing edible oil capsules, the raw score of immediate memory significantly increased by 11.9 on an average and that of delayed memory significantly increased by 18.1 on an average.

Thus, it has been demonstrated for the first time that the intake of an arachidonic acid- and docosahexaenoic acid-containing edible oil can ameliorate reduced higher brain functions resulting from organic brain lesions.

### Example 4

### Use in an Oil Infusion

200 g of the arachidonic acid-containing oil (triglyceride) obtained in Example 1, 200 g of a docosahexaenoic acid-containing oil (triglyceride) purified from fish oil, 48 g of purified egg yolk lecithin, 20 g of oleic acid, 100 g of glycerin and 40 ml of 0.1N sodium hydroxide were added and dispersed by a homogenizer, and then distilled water for injection was added to make 4 liters. This was emulsified using a high-pressure spray-type emulsifying machine to prepare an oil emulsion. After said oil emulsion was dispensed in 200 ml aliquots into plastic bags, they were subjected to high-pressure steam sterilization at 121° C. for 20 minutes to prepare an oil infusion.

### Example 5

### Use in a Juice

Two grams of β-cyclodextrin was added to 20 ml of an aqueous solution of 20% ethanol, to which 100 mg of a mixture (vitamin E being blended to 0.05%) of the arachidonic acid-containing oil (triglyceride) obtained in Example 1 and a docosahexaenoic acid-containing oil (triglyceride) purified from fish oil was added, and incubated at 50° C. for 2 hours. After cooling to room temperature (for about 1 hour), it was further incubated, while stirring at 4° C. for 10 hours. The precipitate formed was recovered by centrifugation, washed in n-hexane, and lyophilized to obtain 1.8 g of a cyclodextrin inclusion compound containing an arachidonic acid- and docosahexaenoic acid-containing triglyceride. One gram of this powder was homogenously mixed with 10 L of a juice to prepare a juice containing an arachidonic acid- and docosahexaenoic acid-containing triglyceride.

### Example 6

### Preparative Example of Capsules Comprising a Compound Having Arachidonic Acid as a Constituent Fatty Acid

Water was added to 100 parts by weight of gelatin and 35 parts by weight of food additive glycerin and dissolved at 50-60° C. to prepare a gelatin coat with a viscosity of 2000 cp. Then, vitamin E oil was mixed at 0.05% by weight in the arachidonic acid-containing oil (triglyceride) obtained in Example 1 to prepare content 2. To a 99% arachidonic acid ethyl ester prepared in Example 1, vitamin E oil was mixed to 0.05% by weight to prepare content 3. Using these contents 2-3, capsules were formed and dried according to standard methods to prepare soft capsules containing 180 mg of the content per capsule.

1-20. (canceled)

21. A method of ameliorating reduced higher brain functions resulting from organic brain lesions comprising administering an effective amount of a composition to a subject in need thereof, wherein the composition comprises arachidonic acid and/or a compound having arachidonic acid as a constituent fatty acid and docosahexaenoic acid and/or a compound having docosahexaenoic acid as a constituent fatty acid, wherein the weight ratio of arachidonic acid to docosahexaenoic acid in the pharmaceutical composition is about 1:1.

22. The method of claim 21, wherein the compound having arachidonic acid as a constituent fatty acid is an alcohol ester of arachidonic acid, or a triglyceride, a phospholipid or a glycolipid wherein part or all of the constituent fatty acids is arachidonic acid, and wherein the compound having docosahexaenoic acid as a constituent fatty acid is an alcohol ester of docosahexaenoic acid, or a triglyceride, a phospholipid or a glycolipid in which part or all of the constituent fatty acids is docosahexaenoic acid.

23. The method of claim 21, wherein the compound having arachidonic acid or docosahexaenoic acid as a constituent fatty acid is a triglyceride in which part or all of the constituent fatty acid is arachidonic acid or docosahexaenoic acid, and wherein the weight ratio of arachidonic acid or docosahexaenoic acid in the triglyceride relative to the total fatty acids constituting the triglyceride is 5% or more.

24. The composition of claim 21, wherein the compound having arachidonic acid or docosahexaenoic acid as a constituent fatty acid is a phospholipid in which part or all of the constituent fatty acid is arachidonic acid or docosahexaenoic acid, and wherein the weight ratio of arachidonic acid or docosahexaenoic acid in the phospholipid relative to the total fatty acids constituting the phospholipid is 5% or more.

25. A method of ameliorating reduced higher brain functions resulting from organic brain lesions comprising administering an effective amount of a composition to a subject in need thereof, wherein the composition comprises a compound having arachidonic acid and docosahexaenoic acid as constituent fatty acids.

26. The method of claim 25, wherein the compound having arachidonic acid and docosahexaenoic acid as constituent fatty acids is a triglyceride, a phospholipid or a glycolipid in which part or all of the constituent fatty acids are arachidonic acid and docosahexaenoic acid.

27. The method of claim 21, wherein the organic brain lesions are caused by ischemic stroke such as cerebral infarction and transient cerebral ischemic attack.

28. The method of claim 21, wherein the organic brain lesions are caused by hemorrhagic stroke such as cerebral hemorrhage and subarachnoid hemorrhage.

29. The method of claim 21, wherein the organic brain lesions are caused by lesional diseases such as cerebral concussion and cerebral contusion.

30. The method of claim 21, wherein the composition is a food composition or a pharmaceutical composition.

31. The method of claim 30, wherein the food composition is a functional food, a nutrient supplement, a food for specified health uses, or a food for the elderly people.

32. The method of claim 30, wherein the pharmaceutical composition is an enteral nutrient, a powder, a granule, a troche, an oral liquid, a suspension, an emulsion, a syrup, or a capsule.
